(19) United States
(12) Patent Application Publication
(10) Pub. No.: US 2007/0201031 A1
(43) Pub. Date: Aug. 30, 2007

(54) Optical Blood Pressure and Velocity Sensor

(75) Inventors: Noel Axelrod, Bnei Brak (IL); Eran Ofek, Bnei Brak, IL (US)

Correspondence Address: EDWARD S. SHERMAN, ESQ., 3554 ROUND BARN BLVD., SUITE 303, SANTA ROSA, CA, 95403, US

(73) Assignee: PHYSICAL LOGIC AG, Zug (CH)

(21) Appl. No.: 11/678,612

(22) Filed: Feb. 25, 2007

Related U.S. Application Data
(60) Provisional application No. 60/777,727, filed on Feb. 28, 2006.
(60) Provisional application No. 60/777,715, filed on Feb. 28, 2006.

(51) Int. Cl. G01B 9/02; A61B 6/00

(52) U.S. Cl. 356/477; 356/450; 600/478

(57) ABSTRACT

A single point implantable optical sensor measures in vivo changes in blood pressure and velocity. An optical fiber waveguide in a catheter transmits light to M-Z interferometer. The wave propagation of fluctuating blood pressure in a living organism is measured by recording the time dependence optical signal losses as the wave traverse each leg of the M-Z device. The time lag between the pressure induced transmission losses at each spaced apart leg is used to calculate blood velocity at the location of the sensor. A plurality of the sensors may be distributed along or catheter in communication via a common optical waveguide.

### CROSS REFERENCE TO RELATED APPLICATIONS

The present application claims priority to the U.S. provisional patent application for “Optical and blood pressure and velocity sensor” filed on Feb. 28, 2006 and assigned application Ser. No. 60/777,727, which is incorporated herein by reference.

The present application also claims priority to the U.S. provisional patent application for an “Optical Sensing Catheter System” filed Feb. 28, 2006, and assigned application Ser. No. 60/777,715, which is incorporated herein by reference.

### BACKGROUND OF INVENTION

The present invention relates to methods and an apparatus for the in-vivo measurement pressure and blood flow in patients.

Catheters that include sensors to measure blood flow are well known. U.S. Pat. No. 5,280,786 to Wlodarczyk et al. issued on Jan. 25, 1994 for a Fiberoptic blood pressure and oxygenation sensor deployed on a catheter placed transcutaneously into a blood vessel. A sensing tip of the catheter includes a pressure-sensing element and an oxygen saturation-measuring element.

It is also known that blood flow, or velocity can be measured by Doppler ultrasound methods. For Example, U.S. Pat. No. 6,616,611 to Moehring issued Sep. 9, 2003 for a Doppler ultrasound method and apparatus for monitoring blood flow describes a pulse Doppler ultrasound system and associated methods are described for monitoring blood flow. It has been contemplated that Doppler ultrasound sensors can be placed internally. For example, U.S. Pat. No. 6,704,590 to Haldeman issued Mar. 9, 2004 for a doppler guiding catheter using a piezoelectric sensor or an optical sensor at the tip to show turbulence through a time domain or frequency domain presentation of velocity. The sensor readings can be used to modulate an audible waveform to indicate turbulence. Detecting changes in a blood flow turbulence level is used to assist guiding of the distal end of the flexible shaft.

However, to measure both velocity and blood pressure simultaneously would require multiple transducer elements either on the catheter, or distributed along the catheter. In the former case, of using two transducers at the tip, such a configuration would increase the catheter diameter, and limiting the deployment of the catheter to wider arteries to minimize the potential for the catheter to affect the blood flow and pressure. In the alternative case of distributing transducers along the catheter, such a configuration could also undesirably increase the catheter diameter to accommodate multiple pairs of wires, as well as disperse the transducers such that they no longer provide a measurement representing the pressure and velocity at a single one point. This is important because, depending on the size of the catheter and its placement, it is simultaneously desirable to keep the catheter diameter as small as possible, and obtain both measurements from the tip of the catheter, to provide more representative measurements of blood pressure and blood velocity free from disturbances and errors due to the presence or location of the catheter or its deployment in smaller arteries.

As there is no convenient method to simultaneously measure blood flow and blood pressure with the same or nearby transducers placed in a patient arterial and vascular systems it is a first object of the present invention to provide a means for the simultaneous measurement of blood pressure and blood flow that can be inserted at a desired location to measure such parameters instantaneously.

It is yet another object of the present invention to provide such a sensor device that is easier to integrate with other biomedical devices and transducers.

It is another object of the invention to provide an optical means for precise local measurement of blood pressure and blood flow in a compact device that is smaller in size than that of the prior art.

It is a further objective of the present invention to provide an optical sensing means for blood pressure and blood flow that is sufficiently compatible with blood that it can remain in a patient for a long period of time, and be deployed in smaller veins and/or arteries.

It is also a further objective of the present invention to provide such a device that is capable of a far more accurate local and representative determination of blood pressure and blood flow.

### SUMMARY OF INVENTION

In the present invention, the first object is achieved by providing an elongated sheath, at least one waveguide (such as an optical fiber for example) disposed within said elongated sheath, and a Mach-Zehnder Interferometer (MZI) in optical communication with said waveguide and disposed with a single arm in tactile communication with the environment external to said sheath.

A second aspect of the invention is characterized by the method of providing an MZI in optical communication between a light source and a photodetector and in tactile communication with blood, measuring the time variant attenuation of light from the source as modulated by the MZI under the influence of blood pressure fluctuations, then calculating the instantaneous pressure from the time variant light attenuation and thereafter or at least simultaneously calculating the blood velocity from time difference in the maximum attention associated with the systolic pressure wave traversing the legs of the MZI.

The above and other objects, effects, features, and advantages of the present invention will become more apparent from the following description of the embodiments thereof taken in conjunction with the accompanying drawings.

### BRIEF DESCRIPTION OF DRAWINGS

FIG. 1A is a schematic plan view of an interferometric optical blood pressure sensor

FIG. 1B is a schematic through the MZI portion of the sensor of FIG. 1A, taken at section line B-B.

FIG. 2 is a schematic diagram of the operative principles in the use of the sensor of FIG. 1 to measure both blood pressure and blood velocity.

FIG. 3 is a schematic illustration of the interferometric optical blood pressure sensor as part of a catheter assembly.

FIG. 4A is a schematic illustration of an alternative embodiment of the interferometric optical blood pressure sensor as part of a different catheter now deploying two or more of the MZI devices of FIG. 1 and FIG. 2.

FIG. 4B is a schematic diagram of the operative principles in using the two or more of the MZI devices of FIG. 4A.

### DETAILED DESCRIPTION

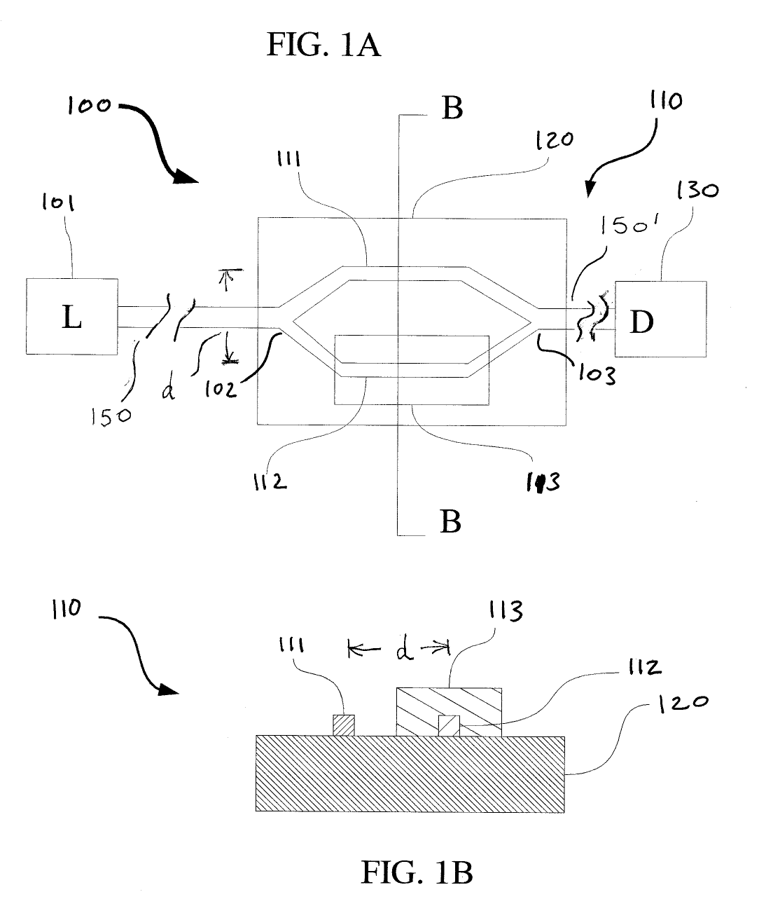

Referring to FIGS. 1 through 5, wherein like reference numerals refer to like components in the various views, there is illustrated therein a new Optical Blood Pressure and Velocity Sensor, generally denominated 100 herein.

A guided wave optical blood pressure sensor can be realized with a balanced Mach-Zehnder waveguide Interferometer (MZI) 110 shown in FIG. 1A. The MZI receives light from a coherent laser source 101 at the left. The light is split by the entry waveguide portion 102 into an upper arm 111 and a lower arm 112. The MZI is mounted on a substrate 120 with the bottom arm 112 protected by a cap or coating 113. Thus, when the MZI portion 110 of device 100 is inserted in fluid communication with the blood stream, pressure is applied only to the upper arm 111, as the lower arm 112 is protected. The applied pressure produces a change in the refractive index due to the photoelastic effect. This in turn modulates the phase of the transmitted wave in the exposed upper arm 111, such that when the light in both arms combines at the exit waveguide portion 103 there is a decrease in optical power in the light transmitted from the laser 101 through the MZI 110 that is received at detector 130.

Preferably, the light from the coherent light source is directed to the MZI 110 via a first optical fiber waveguide segment 150, and is then directed to detector 130 via a second optical fiber waveguide segment 150′. As will be further described in other embodiments, multiple MZI devices 110 can be deployed along a single optical fiber waveguide bus for measuring the blood pressure and velocity at multiple locations along the catheter.

The device 100 of FIG. 1 has many benefits. As multiple physiological measurements can now be made at the tip, or elsewhere on a catheter or related implantable medical device, the small sensor size avoids interference with blood flow. Further, the combined blood flow measurements are useful in the diagnosis of vascular disease and the control of pacemakers and ICD's for example.

In preferred embodiments, the interferometer arms 111 and 112 can be fabricated from polydimethlysiloxane (PDMS) on a PDMS substrate. This material has high optical transmittance, high elasticity and is biocompatible. These properties make it particularly attractive to blood pressure sensor applications.

In terms of the more detailed description of the proposed device, the phase difference induced by the pressure applied to its one arm is

$$Δφ = \frac{2 π}{λ} Δ nl \qquad ( 1 )$$

where l is the arm's length and Δn is the refractive index change that is
Δn=n³ρS (2)
where ρ is the elasto-optic coefficient, and S is the strain S=P/E. In the last formula P is the applied stress and E is the Young module of the material of the waveguide.

The power transmittance of the Mach-Zehnder Interferometer is

$$T = \frac{{I}_{out}}{{I}_{i n}} = {sin}^{2} \frac{Δ φ + {φ}_{0}}{2} = \frac{1}{2} ( 1 - cos ( Δ ϕ + {ϕ}_{0} ) ) \qquad ( 3 )$$

where Iᵢₙ and Iₒᵤₜ is the input and the output intensities and φ₀ if the phase difference between the interferometer arms in absence of pressure. Because Δφ varies linearly with the applied pressure P, the interferometer would have a linear response if its transmittance T varies linearly with Δφ. It can be seen from Eq. (3) that it is not generally true. However, for small variations of Δφ, it is approximately true near

$${φ}_{0} = \frac{π}{2} ,$$

as can be seen by substituting this value of φ₀ to Eq. (3) to get

$$T = \frac{1}{2} ( 1 + sin Δ φ ) \approx \frac{1}{2} ( 1 + Δ φ ) for \vert Δφ \vert << π.$$

Finally we get from Eq.(2) and Eq. (3)

$$Δφ = \frac{2 π}{λ} {n}^{3} p \frac{P}{E} l . \qquad ( 4 )$$

Let us define a pressure P_{π} as a pressure at which Δφ=π. From Eq. (4)

$${P}_{π} = \frac{λ E}{2 l ρ {n}^{3}} \qquad ( 5 )$$

As an example, we calculate P_{π} for a PDMS sensor with arm length l=1 cm at λ=600 nm. The parameters of PDMS are the following: E=750 kPa, n=1.45, ρ=0.1. Substituting these values to Eq. (5) we find P_{π}=67 Pa.

As a rule of thumb, sensitivity of the interferometer sensor is about P_{π}/100. Therefore, the sensitivity of the PDMS interferometer is as high as 0.7 Pa.

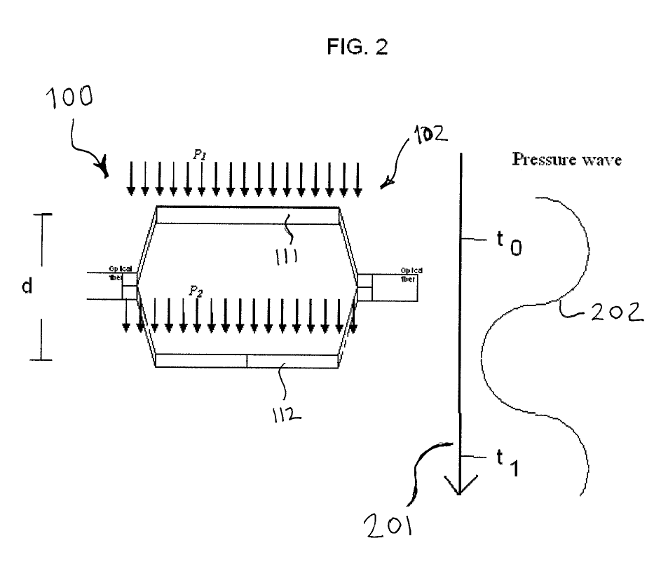

As schematically illustrated in FIG. 2, the Mach-Zehnder waveguide Interferometer, described above and with respect to FIGS. 1A and 1B, can be used also for measuring velocity of the blood in blood vessels. During the systolic heart cycle a pressure wave is produced. The arrow 201 indicates direction of pressure wave propagation, shown in a simplified sinusoidal shaped schematic of the pressure amplitude to the right of arrow 201 as shape 202. At time t₀ the pressure wave 202 has a peak at arm 111. Whereas at time t₁ the peak, traveling at the velocity of the blood, ν, has now progressed past arm 111 to the second arm 112.

The velocity can be measured by measuring the time needed to the pressure wave to propagate from one arm of the interferometer to another. Dividing this time on the distance, d, between the interferometer arms, one then gets the velocity of front wave propagation:

$$v = \frac{d}{( {t}_{1} - {t}_{0} )} \qquad ( 6 )$$

Accordingly, another embodiment of the invention is the method of use in which the light is measured by the detector as a function of time. In the next step in this method the instantaneous pressure is calculated from light attenuation according to Eq. (5). Next, the velocity is calculated from the time difference in the maximum attention associated with the systolic pressure wave traversing the arms of the MZI 110.

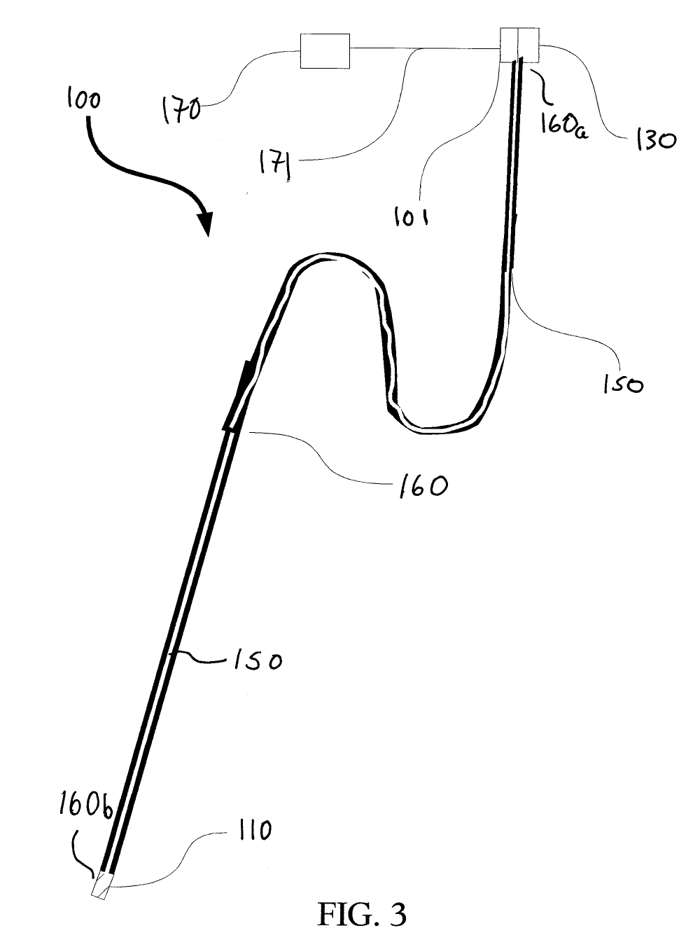

In FIG. 3, the device 100 includes a laser 101 and a detector 130 in optical communication with the MZI device in FIG. 1 via an optical fiber 150. The optical fiber is covered by or forms the core of a catheter or cannula device 160 for insertion in a vein or artery, or any other location where it is desirable to measure at least one of fluid pressure and velocity. The laser 101 and detector 130 may be deployed at the proximal end 160a of the catheter with the MZI device deployed at the distal end 160b to be inserted into the patient. The laser and detector are in signal communication with a controller and data processing unit 170 via cabling or signal carrier lines 171 for carrying out the calculations according to the various embodiment of the invention disclosed herein.

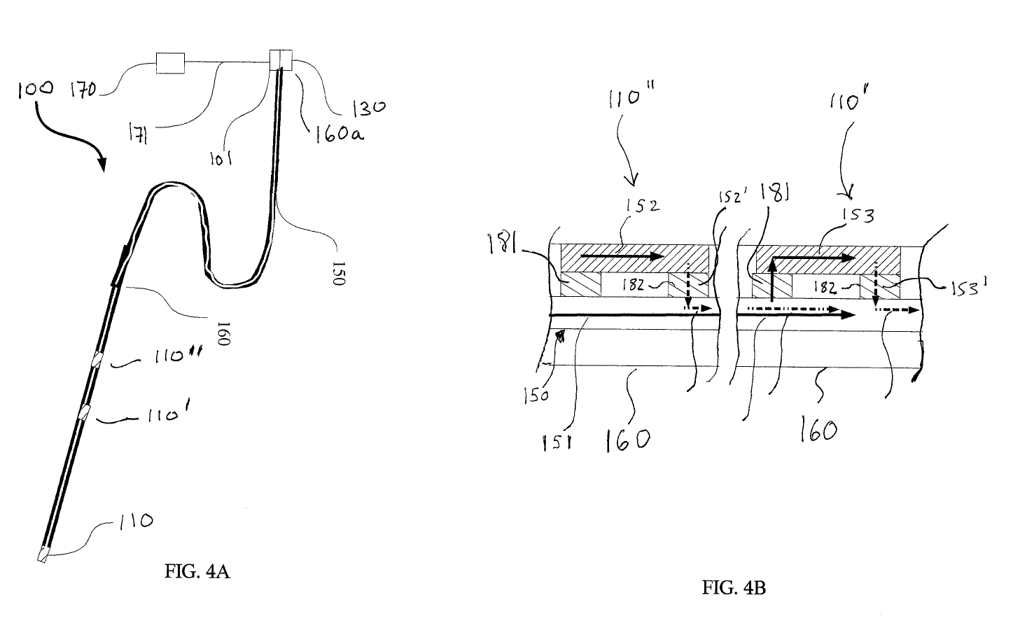

In FIGS. 4A and 4B, the device 100 includes a laser 101 and a detector 130 in optical communication with multiple MZI devices, 100′ and 100″, along with device 110 at the catheter tip 160b. The multiple MZI devices, 110′ and 110″ and 110 are preferably in common connection via an optical fiber 150. As in FIG. 3, the optical fiber is at least partially covered by or forms the core of a catheter or cannula device 160 for insertion in a vein or artery, or other location where it is desirable to measure at least one of fluid pressure and velocity. FIG. 4B illustrates schematically further details of the common connection via an optical fiber 150. Each of the MZI devices 110′ and 110″ receives light from optical fiber 150, the light beam propagating in optical fiber 150 being shown as arrow 151, via a coupler 181.

Most preferably, coupler 181 is an optical de-multiplexer and coupler 182 is an optical multiplexer such that each of devices 110 is separately addresses by a different wavelength of light within light beam 151. Accordingly, the intensity of light of each separate wavelength can be analyzed by a detector to ultimately measure the blood pressure and/or blood velocity at the respective location of each MZI device 110′ and 110″. Thus, light modulated in intensity by the action of blood on the exposed arm of the MZI device is then coupled back into optical fiber 150 by a different multiplexer 182 associated with each MZI. Each multiplexer/de-multiplexer either combines or splits off a distinct wavelength of light to interrogate a distinct MZI as a discrete transducer. Thus, arrow 152′ now indicates light of a specific wavelength modulated by MZI 110′ exiting multiplexer 182 and then propagating as part of light beam 151. Likewise, arrow 152″ now indicates light modulated by MZI 110″ of a different wavelength (than that exiting MZI 110′), exiting the multiplexer 182 associated with MZI 110″ and co-propagating as part of light beam 151.

Such means for wavelength division multiplexing described in the preceding paragraph are well known in the field of optical fiber communication systems. However, the inventive arrangement of multiple MZI devices along catheter 160 as shown in FIGS. 4A and 4B permits a relatively large number of precise measurements to be taken of blood pressure and velocity, yet at the same time maintaining a relatively small catheter diameter.

It will be recognized by one of ordinary skill in the art that there are numerous alternative means to optically couple the MZI in optical communication with the laser and detector, such as for example light is optionally returned to the photodetector either by a mirror means or via an optical loop. Additionally, a wide variety of multiplexing/de-multiplexing optical couplers are available as couplers 181 and 182 as shown in FIG. 4B. FIG. 5 illustrates one such embodiment wherein each leg of the MZI 110 terminates in a mirror. Thus, light entering leg 111 is reflected by mirror 501 and light entering leg 112, protected from the blood pressure by cap or coating 113, is reflected by mirror 502 such that when the light reflected by both legs combines in optical fiber 150, there is a modulation of intensity due to the photoelastically induced phase modulation occurring in leg 111.

Further, it should be appreciated that other embodiments of the invention embrace alternative types of cannulae, catheters or medical devices in which the sensor is implanted on or communicates with another device, such as pacemakers, ECD's and stents.

While the invention has been described in connection with a preferred embodiment, it is not intended to limit the scope of the invention to the particular form set forth, but on the contrary, it is intended to cover such alternatives, modifications, and equivalents as may be within the spirit and scope of the invention as defined by the appended claims.

1. A device comprising:
a) an elongated sheath,
b) at least one waveguide disposed within said elongated sheath,
c) at least one Mach-Zehnder Interferometer (MZI) in optical communication with said waveguide and disposed with a single arm in tactile communication with the environment external to said sheath,
d) means to receive and detect fluctuations in light intensity arising from the transmission of external pressure fluctuations to the MZI.

2. The device of claim 1 wherein the detector is in optical communication with the MZI via the same waveguide via a mirror.

3. The device of claim 1 wherein the detector is in optical communication with the MZI via another waveguide forming an optical loop.

4. The device of claim 1 further comprising a light source to illuminate the waveguide and MZI.

5. The device according to claim 4 wherein the light source is a multiple wavelength light source.

6. The device according to claim 5 wherein the light source is a laser.

7. A device comprising,
a) an elongated sheath,
b) at least one waveguide disposed within said elongated sheath,
c) at least one Mach-Zehnder Interferometer (MZI) in optical communication with said waveguide and disposed with a single arm in tactile communication with the environment external to said sheath,
d) a detector in optical communication with the MZI to detect fluctuations in light intensity arising from the transmission of external pressure fluctuations to the MZI.

8. The device of claim 7 wherein the detector is in optical communication with the MZI with the same waveguide used to illuminate the MZI via a mirror.

9. The device of claim 7 wherein the detector is in optical communication with the MZI via another waveguide forming an optical loop.

10. The device of claim 7 further comprising a light source to illuminate the waveguide and MZI.

11. The device of claim 10 wherein the light source is a multiple wavelength light source.

12. The device of claim 11 wherein the light source is a laser.

13. The device of claim 11 wherein the detector is a demultiplexer.

14. The device of claim 7 wherein at least one of the interferometer arms is comprised of polydimethlysiloxane (PDMS)

15. The device of claim 13 wherein the interferometer arms are disposed on a PDMS substrate.

16. A method of measuring at least one of blood pressure and velocity, the method comprising the steps of:
a) providing an MZI in optical communication between a light source and photodetector and in tactile communication with blood,
b) measuring the time variant attenuation of light from the source as modulated by MZI under the influence of blood pressure fluctuations,
c) calculating the instantaneous pressure from the time variant light attenuation.

17. The method according to claim 16 further comprising the step of calculating the blood velocity from the time difference in the maximum attention associated with the systolic pressure wave traversing the legs of the MZI.

18. The method of claim 16 wherein at least one MZI is disposed in optical communication with an optical fiber for receiving the light.

19. The method of claim 18 wherein the optical fiber is illuminated with a multiple wavelength source to illuminate a plurality of different MZI's disposed along the optical fiber.

20. The method of claim 19 wherein a detector for measuring the time variant attenuation of light demuliplexes the multiple wavelengths received from the different MZI's.
